(19) United States
(12) Patent Application Publication
(10) Pub. No.: US 2022/0090179 A1
(43) Pub. Date: Mar. 24, 2022

(54) COMPOSITIONS AND METHODS FOR DETECTING AN ABASIC SITE

(71) Applicant: ILLUMINA, INC., San Diego, CA (US)

(72) Inventors: Jie Mao, San Diego, CA (US); Randall Smith, San Marcos, CA (US)

(73) Assignee: ILLUMINA, INC., San Diego, CA (US)

(21) Appl. No.: 17/472,038

(22) Filed: Sep. 10, 2021

Related U.S. Application Data
(60) Provisional application No. 63/077,119, filed on Sep. 11, 2020.

(51) Int. Cl. C12Q 1/6834; G01N 21/64; C12Q 1/6816

(52) U.S. Cl. CPC C12Q 1/6834; G01N 21/6428; G01N 2021/6497; G01N 2021/6495; C12Q 1/6816

(57) ABSTRACT

A method for detecting an abasic site is provided. The method may include flowing a solution over a substrate having a plurality of oligonucleotides coupled thereto. At least one of the oligonucleotides includes an abasic site. The solution may include a fluorophore coupled to a reactive group. The method may include reacting the reactive group with the abasic site to couple the fluorophore to the abasic site; and detecting the abasic site using fluorescence from the fluorophore.

### CROSS-REFERENCE TO RELATED APPLICATIONS

This application claims the benefit of U.S. Provisional Patent Application No. 63/077,119, filed Sep. 11, 2020 and entitled “Compositions and Methods for Detecting an Abasic Site,” the entire contents of which are incorporated by reference herein.

### SEQUENCE LISTING

10001.11 The instant application contains a Sequence Listing which has been submitted electronically in ASCII format and is hereby incorporated by reference in its entirety. Said ASCII copy, created on Sep. 7, 2021, is named IP-2017-US_SL.txt and is 707 bytes in size.

### BACKGROUND

Cluster amplification is an approach to amplifying polynucleotides, for example for use in genetic sequencing. Target polynucleotides are captured by oligonucleotide primers (e.g., P5 and P7 primers) coupled to a substrate surface in a flowcell, and form “seeds” at random locations on the surface. Cycles of amplification are performed to form clusters of amplicons on the surface around each seed, e.g., using “bridge amplification.”

### SUMMARY

Examples provided herein are related to detecting an abasic site. Compositions and methods for performing such detection are disclosed.

Provided in some examples herein is a method for detecting an abasic site. The method may include flowing a solution over a substrate having a plurality of oligonucleotides coupled thereto. At least one of the oligonucleotides may include an abasic site. The solution may include a fluorophore coupled to a reactive group. The method may include reacting the reactive group with the abasic site to couple the fluorophore to the abasic site; and detecting the abasic site using fluorescence from the fluorophore.

In some examples, the abasic site is generated by damage to the oligonucleotide.

In some examples, nucleotide bases adjacent to the abasic site may inhibit non-radiative energy dissipation from the respective fluorophore coupled to the abasic site.

In some examples, the fluorophore may include a molecular rotor dye that includes π-conjugated components separated by a rotatable C—C bond. In some examples, nucleotide bases adjacent to the abasic site may restrict rotation of the C—C bond and may align the π-conjugated components with one another.

In some examples, the molecular rotor dye coupled to the reactive group is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange).

In some examples, the fluorophore coupled to the reactive group is selected from the group consisting of:

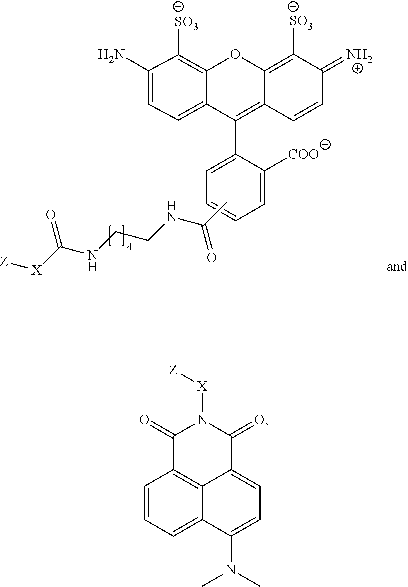

where X is a linker and Z is the reactive group.

In some examples, the abasic site includes an aldehyde. In some examples, the reactive group includes a hydroxylamine group. In some examples, the reactive group includes a hydrazine group. In some examples, reacting the reactive group with the abasic site forms an oxime linkage.

Provided in some examples herein is a composition. The composition may include a substrate having a plurality of oligonucleotides coupled thereto. At least one of the oligonucleotides may include an abasic site. The composition may include fluorophore coupled to the abasic site. The abasic site may be detectable using fluorescence from the fluorophore.

In some examples, the abasic site is generated by damage to the oligonucleotide.

In some examples, nucleotide bases adjacent to the abasic site inhibit non-radiative energy dissipation from the respective fluorophore coupled to the abasic site.

In some examples, the fluorophore may include a molecular rotor dye that includes π-conjugated components separated by a rotatable C—C bond. In some examples, nucleotide bases adjacent to the abasic site restrict rotation of the C—C bond and align the π-conjugated components with one another. In some examples, the molecular rotor dye coupled to the reactive group is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange).

In some examples, the fluorophore coupled to the reactive group is selected from the group consisting of:

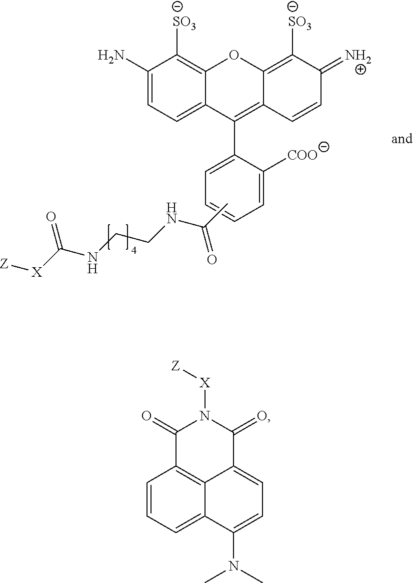

where X is a linker and Z is the reactive group.

In some examples, the abasic site includes an aldehyde. In some examples, the reactive group includes a hydroxylamine group. In some examples, the reactive group includes a hydrazine group. In some examples, reacting the reactive group with the abasic site forms an oxime linkage.

Provided in some examples herein is a method. The method may include preparing a solution that includes (i) glycosylases, (ii) oligonucleotides, and (iii) fluorophores coupled to reactive groups. The method may include generating, using the glycosylases, abasic sites in the oligonucleotides in the solution. The method may include reacting the reactive groups with the abasic sites to couple the fluorophores to the abasic sites. The method may include measuring activity of the glycosylases using fluorescence from the fluorophores coupled to the abasic sites. The method may include using the glycosylases in a sequencing-by-synthesis operation.

In some examples, nucleotide bases adjacent to the abasic site may inhibit non-radiative energy dissipation from the respective fluorophore coupled to the abasic site.

In some examples, the fluorophore may include a molecular rotor dye that includes π-conjugated components separated by a rotatable C—C bond. In some examples, nucleotide bases adjacent to the abasic site may restrict rotation of the C—C bond and may align the π-conjugated components with one another. In some examples, the molecular rotor dye coupled to the reactive group is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange).

In some examples, the fluorophore coupled to the reactive group may be selected from the group consisting of:

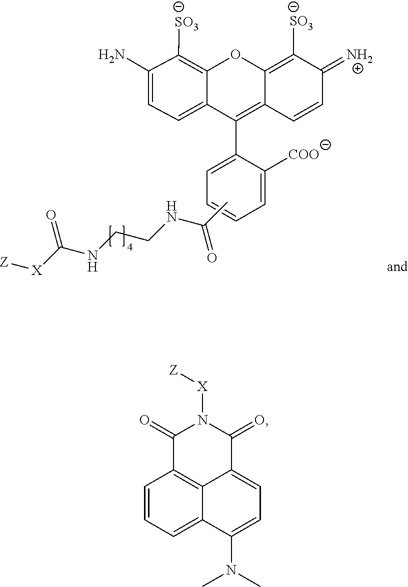

where X is a linker and Z is the reactive group.

In some examples, the abasic site includes an aldehyde. In some examples, the reactive group includes a hydroxylamine group. In some examples, the reactive group includes a hydrazine group. In some examples, reacting the reactive group with the abasic site forms an oxime linkage.

Provided in some examples herein is a solution for measuring glycosylase activity. The solution may include (i) glycosylases, (ii) oligonucleotides, and (iii) fluorophores coupled to reactive groups. The oligonucleotides may include abasic sites generated by the glycosylases in the solution. The fluorophores may be coupled to the abasic sites. The abasic sites may be detectable using fluorescence from the fluorophores.

In some examples, nucleotide bases adjacent to the abasic site may inhibit non-radiative energy dissipation from the respective fluorophore coupled to the abasic site.

In some examples, the fluorophore may include a molecular rotor dye that includes π-conjugated components separated by a rotatable C—C bond. In some examples, nucleotide bases adjacent to the abasic site may restrict rotation of the C—C bond and may align the K-conjugated components with one another. In some examples, the molecular rotor dye coupled to the reactive group is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange).

In some examples, the fluorophore coupled to the reactive group is selected from the group consisting of:

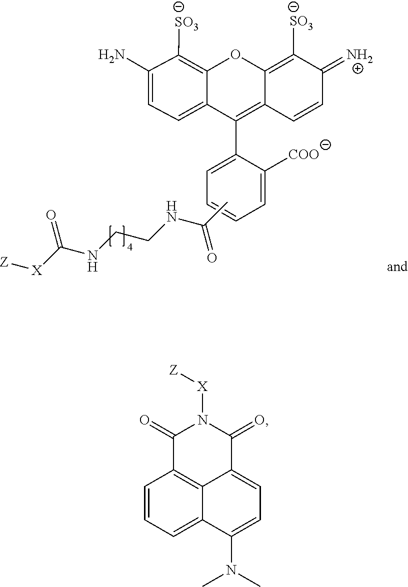

where X is a linker and Z is the reactive group.

In some examples, the abasic site includes an aldehyde. In some examples, the reactive group includes a hydroxylamine group. In some examples, the reactive group includes a hydrazine group. In some examples, reacting the reactive group with the abasic site forms an oxime linkage.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate an example composition for detecting an abasic site, such as caused by damage to an oligonucleotide.

FIG. 2 schematically illustrates operations in an example method for detecting an abasic site, such as caused by damage to an oligonucleotide.

FIGS. 3A-3C schematically an example composition for measuring an amount of abasic sites, such as for measuring glycosylase activity.

FIG. 4 schematically illustrates operations in an example method for measuring an amount of abasic sites, such as for measuring glycosylase activity.

### DETAILED DESCRIPTION

Examples provided herein are related to detecting an abasic site. Some examples provided herein are related to detecting damage to oligonucleotides, or to measuring glycosylase activity. Compositions and methods for performing such detection and measurement are disclosed.

The abasic site herein may refer to a DNA abasic site. DNA abasic sites (also referred to as apurinic/apyrimidinic sites, or “AP”) may be generated intentionally, e.g., by DNA glycosylases. For example, glycosylases may be used in one or more sequencing-by-synthesis (“SBS”) operations, such as to linearize amplicons generated using “bridge amplification.” Illustratively, Uracil-DNA glycosylase (UDG) may be used to generate abasic sites at dU bases, and the abasic sites then may be processed by an endonuclease to generate cuts in the phosphodiester backbone and thus linearize the amplicons. DNA abasic sites also may be generated unintentionally, e.g., by damage to the DNA such as from exposure to an acidic medium. Detecting damage to oligonucleotides, such as primers, may be useful because abasic sites that are generated by such damage may be inadvertently cleaved in a later operation.

Additionally, measuring activity of glycosylases may be useful because if the glycosylases generate abasic sites in amplicons at an insufficient rate, then the amplicons may be insufficiently linearized which may detrimentally affect subsequent SBS operations.

As provided herein, the intentional or unintentional generation of abasic sites (e.g., by glycosylase activity, or by damage) may be detected by coupling fluorophores to the abasic sites. For example, the fluorophores may be coupled to reactive moieties that react with the abasic sites and thus couple the fluorophores to the abasic sites. Illustratively, abasic sites may form aldehydes that, when reacted with reactive moieties, such as hydroxylamines or hydrazines, form oximes via which fluorophores are coupled to the abasic sites. In some examples, fluorescence from the fluorophores may be turned on or enhanced when the fluorophores are coupled to the abasic sites. Illustratively, nucleotide bases adjacent to the abasic site may inhibit non-radiative energy dissipation from the fluorophore, and thus may enhance the intensity of fluorescence from the fluorophore or even cause the fluorophore to detectably fluoresce only once coupled to the abasic site.

First, some terms used herein will be briefly explained. Then, some example compositions and example methods for detecting abasic sites, such as caused by damage to oligonucleotides, or for measuring glycosylase activity, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to +0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” refers to a polynucleotide to which nucleotides may be added via a free 3′ OH group. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer. A primer may be coupled to a substrate.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned, covalently-linked gel such as poly(N-(5-azidoacetamidylpentyl) acrylamide-co-acrylamide) (PAZAM). The process creates gel pads used for sequencing that may be stable over sequencing runs with a large number of cycles. The covalent linking of the polymer to the wells may be helpful for maintaining the gel in the structured features throughout the lifetime of the structured substrate during a variety of uses. However in many examples, the gel need not be covalently linked to the wells. For example, in some conditions silane free acrylamide (SFA) which is not covalently attached to any part of the structured substrate, may be used as the gel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, Calif.).

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

As used herein, the term “glycosylase” refers to an enzyme that hydrolyzes a glycosyl compound. In some examples, the glycosyl compound that the glycosylase hydrolyzes may be included in a polynucleotide. The polynucleotide may be single-stranded or double-stranded.

DNA and RNA are non-limiting examples of polynucleotides with which a glycosylase may be used in a manner such as provided herein. In some examples, glycosylases that may be used in a manner such as provided herein are “monofunctional” which is intended to mean that they lack additional activity beyond glycosylase activity. In comparison, “bifunctional” DNA glycosylases also may cut the phosphodiester bond of DNA. The “activity” of a glycosylase, as used herein, may express the rate at which the glycosylase hydrolyzes glycosyl compounds as a function of time.

Glycosylases include DNA glycosylases, which recognize and remove DNA bases that are damaged or mispaired by hydrolyzing the N-glycosidic bond between that base and the deoxyribose, thus generating an abasic site that includes a hemiacetal group which is equilibrium with an aldehyde group. Nonlimiting examples of monofunctional glycosylases include Uracil-DNA glycosylase (UDG), which may be used to generate abasic sites at dU bases as may result from cytosine deamination; AlkA/AlkE/Mag1/MPG (N-methyl purine DNA glycosylase) which may be used to generate abasic sites at 3-meA (3-alkyladenine) and hypoxanthine; MutY/mHYH which may be used to generate abasic sites at A:8-oxoG; hSMUG1 which may be used to generate abasic sites at U, hoU (5-hydroxyuracil), hmU (5-hydroxymethyluracil), or fU (5-formyluracil); TDG or MBD4 which may be used to generate abasic sites at T:G mispairings; and AlkC or AlkD which may be used to generate abasic sites at alkypurine.

As used herein, the term “fluorophore” is intended to mean a molecule that emits light at a first wavelength responsive to excitation with light at a second wavelength that is different from the first wavelength. The light emitted by a fluorophore may be referred to as “fluorescence” and may be detected by suitable optical circuitry. In addition to fluorescing, which may be considered to “radiatively” emit energy, a fluorophore may “non-radiatively” dissipate energy, such as via rotation of the molecule or of one or more components of such molecule. Non-radiative energy dissipation may reduce the amount of energy that the fluorophore may use to emit energy radiatively. An example fluorophore is a “molecular rotor dye,” which refers to a fluorophore with a carbon-carbon (“C—C”) single bond rotational axis between two π-conjugated components. When the C—C bond may freely rotate, the π-conjugated components may not align with one another, and as the molecule substantially may not fluoresce. In comparison, when rotation of the C—C bond is restricted in such a manner so as to sufficiently align the π-conjugated components with each other that the π-orbitals of those components overlap with one another and form an extended π-conjugated assembly, the resulting extended π-conjugated assembly may detectably fluoresce at a relatively high intensity as compared to when the x-conjugated components are not aligned.

As used herein, to “detect” fluorescence is intended to mean to receive light from a fluorophore, to generate an electrical signal based on the received light, and to determine, using the electrical signal, that light was received from the fluorophore. Fluorescence may be detected using any suitable optical detection circuitry, which may include an optical detector to generate an electrical signal based on the light received from the fluorophore, and electronic circuitry to determine, using the electrical signal, that light was received from the fluorophore. As one example, the optical detector may include an active-pixel sensor (APS) including an array of amplified photodetectors configured to generate an electrical signal based on light received by the photodetectors. APSs may be based on complementary metal oxide semiconductor (CMOS) technology known in the art. CMOS-based detectors may include field effect transistors (FETs), e.g., metal oxide semiconductor field effect transistors (MOSFETs). In particular examples, a CMOS imager having a single-photon avalanche diode (CMOS-SPAD) may be used, for example, to perform fluorescence lifetime imaging (FLIM). In other examples, the optical detector may include a photodiode, such as an avalanche photodiode, charge-coupled device (CCD), cryogenic photon detector, reverse-biased light emitting diode (LED), photoresistor, phototransistor, photovoltaic cell, photomultiplier tube (PMT), quantum dot photoconductor or photodiode, or the like. The optical detection circuitry further may include any suitable combination of hardware and software in operable communication with the optical detector so as to receive the electrical signal therefrom, and configured to detect the fluorescence based on such signal, e.g., based on the optical detector detecting light from the fluorophore. For example, the electronic circuitry may include a memory and a processor coupled to the memory. The memory may store instructions for causing the processor to receive the signal from the optical detector and to detect the fluorophore using such signal. For example, the instructions can cause the processor to determine, using the signal from the optical detector, that fluorescence is emitted within the field of view of the optical detector and to determine, using such determination, that a fluorophore is present.

To “measure” fluorescence is intended to mean to determine a relative or absolute amount of the fluorescence that is detected. For example, the amount of fluorescence may change as a function of time, and changes in the amount of fluorescence may be measured relative to the initial amount of fluorescence, or as an absolute amount of fluorescence. Illustratively, the amount of abasic sites in a plurality of oligonucleotides may change as a function of time, e.g., responsive to action by a glycosylase, and fluorophores may be coupled to the abasic sites. The amount of fluorescence from the plurality of fluorophores may be correlated to the amount of abasic sites, and to activity of the glycosylase. For example, the memory of the electronic circuitry described above may store instructions causing the processor to monitor the level of the electrical signal at one or more times, and to correlate such level(s) to an amount of abasic sites or to an activity of the glycosylase.

Compositions and Methods for Detecting Abasic Sites, Such as Caused by Damage to Oligonucleotides

Some examples provided herein relate to methods for detecting damage to oligonucleotides. For example, oligonucleotides may be coupled to substrates, e.g., within flowcells, for use as primers for generating clusters of amplicons on which SBS operations are to be performed. If the oligonucleotides are stored improperly (e.g., at too high a temperature, or for too long), then at least some of the oligonucleotides may be expected to be damaged, causing generation of at least one abasic site. Such abasic site(s) may be detected by respectively coupling a fluorophore thereto.

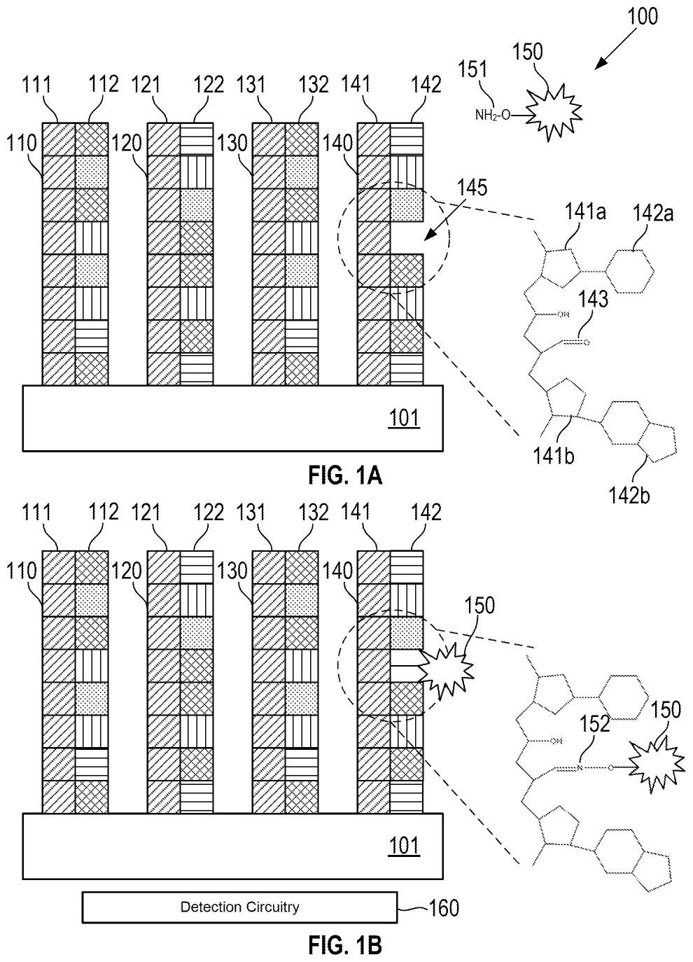

For example, FIGS. 1A-1B schematically illustrate an example composition for detecting an abasic site, such as caused by damage to an oligonucleotide. Composition 100 illustrated in FIG. 1A includes substrate 101 having a plurality of oligonucleotides 110, 120, 130, 140 coupled thereto. In the illustrated example, each of oligonucleotides 110, 120, 130, 140 is single-stranded, although it will be appreciated that the oligonucleotides instead may be single-stranded.

For example, oligonucleotide 110 includes sugar-phosphate backbone 111 and bases 112; oligonucleotide 120 includes sugar-phosphate backbone 121 and bases 122; oligonucleotide 130 includes sugar-phosphate backbone 131 and bases 132; and oligonucleotide 140 includes sugar-phosphate backbone 141 and bases 142. Oligonucleotides 110, 120, 130, 140 may include primers coupled to the surface of substrate 101. In a manner such as suggested by the differently filled boxes in FIG. 1A, the bases 112 of oligonucleotide 110 may have the same sequence as the bases 132 of oligonucleotide 130, and the bases 122 of oligonucleotide 120 may have the same sequence as the bases 142 of oligonucleotide 140 (and a different sequence than the bases of oligonucleotides 110, 130). In one nonlimiting, purely illustrative example, oligonucleotides 110, 130 are P5 capture primers, and oligonucleotides 120, 140 are P7 capture primers. P5 capture primers, which are commercially available from Illumina, Inc. (San Diego, Calif.) have the sequence 5′-AATGATACGGCGACCACCGA-3′ (SEQ ID NO: 1). P7 capture primers, which also are commercially available from Illumina, Inc., have the sequence 5′-CAAGCAGAAGACGGCATACGA-3′ (SEQ ID NO: 2). However, it will be appreciated that the bases of the oligonucleotides may have any suitable sequence or sequences.

At least one of the oligonucleotides may include an abasic site, which may have been generated by damage to that oligonucleotide. Illustratively, one of the bases 142 of oligonucleotide 140 is missing at abasic site 145. As shown in the inset of FIG. 1A, abasic site 145 may include aldehyde 143, and may have a first nucleotide including sugar 141a and (illustratively) pyrimidine base 142a, and a second nucleotide including sugar 141b and (illustratively) purine base 142b, adjacent thereto.

As illustrated in FIG. 1A, composition 100 may include fluorophore 150 that may be coupled to abasic site 145. For example, fluorophore 150 may be coupled to reactive group 151 that may react with abasic site 145 so as to couple fluorophore 150 to abasic site 145 in a manner such as shown in FIG. 1B. Nonlimiting examples of reactive group 151 include hydroxylamine and hydrazine. For example, as shown in the inset of FIG. 1B, hydroxylamine 151 reacts with aldehyde 143 to form oxime linkage 152 via which fluorophore 150 is coupled to abasic site 145. Abasic site 145 may be detectable using fluorescence from the fluorophore, e.g., using suitable detection circuitry 160.

It will be appreciated that fluorophore 150 may include any suitable fluorophore. Nucleotide base(s) adjacent to abasic site 145 may reduce or inhibit nonradiative energy dissipation from fluorophore 150 coupled to that abasic site. For example, fluorophore 150 may include a molecular rotor dye comprising π-conjugated components separated by a rotatable C—C bond. Nucleotide bases 142a, 142b adjacent to abasic site may restrict rotation of the C—C bond and align the π-conjugated components with one another. Such restriction of rotation, when coupled to abasic site 145, may enhance fluorescence of fluorophore 150 as compared to when in solution, or even may cause fluorophore 150 to begin to fluoresce. In some examples, the molecular rotor dye coupled to the reactive group 151 is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange). An example structure for CCVJ coupled to reactive group Z by linker X is:

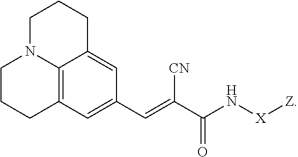

An example structure for DFHBI coupled to reactive group Z is:

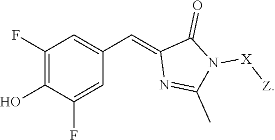

An example structure for thiazole orange coupled to reactive group Z is:

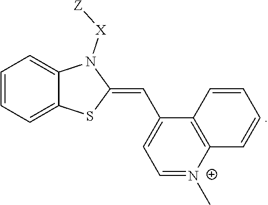

In nonlimiting examples, Z is hydroxylamine (—O—NH₂). In other nonlimiting examples, Z is hydrazine (—NH—NH₂). Z may react with aldehyde 143 so as to form an oxime linkage between fluorophore 150 and abasic site 145.

It will be appreciated that any suitable fluorophore other than a molecular rotor dye suitably may be coupled to a reactive group that may be coupled to an abasic site. Illustratively, the fluorophore coupled to the reactive group 151 may be selected from the group consisting of an Alexa Fluor dye and a 1,8-naphthalene diimide. Alexa Fluor dyes are commercially available from ThermoFisher Scientific (Waltham, Mass.). In one non-limiting example, the Alexa Fluor dye is Alexa Fluor 488. In one non-limiting example, the 1,8-naphthalene diimide is 6-dimethylamino)-2-methyl-1H-benzo[de]isoquinoline-1,3(2H)-dione (NP2). An example structure for Alexa Fluor 488 coupled by linker X to reactive group Z is:

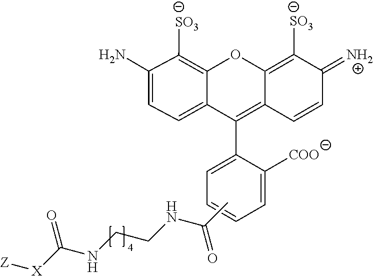

(2-(6-amino-3-iminio-4,5-disulfonato-3H-xanthen-9-yl)-4-((2-(aminooxy)ethyl)carbamoyl)benzoate). An example structure for 6-dimethylamino)-2-methyl-1H-benzo[de]isoquinoline-1,3(2H)-dione (NP2) coupled by linker X to reactive group Z is:

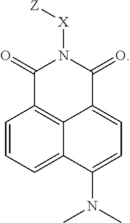

In nonlimiting examples, Z is hydroxylamine (—O—NH₂). In other nonlimiting examples, Z is hydrazine (—NH—NH₂). Z may react with aldehyde 143 so as to form an oxime linkage between fluorophore 150 and abasic site 145.

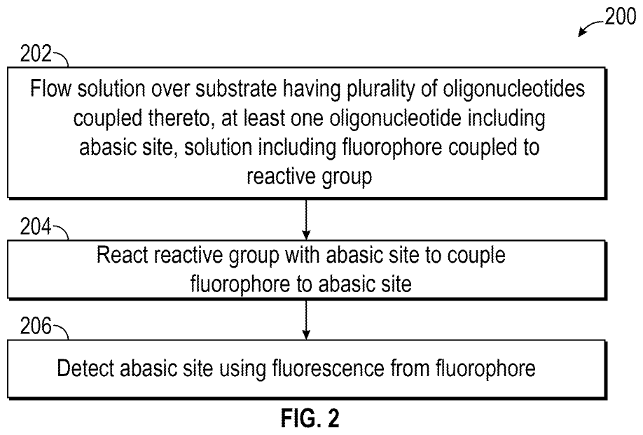

FIG. 2 schematically illustrates an example method for detecting an abasic site, such as caused by damage to an oligonucleotide. Method 200 illustrated in FIG. 2 may include flowing a solution over a substrate having a plurality of oligonucleotides coupled thereto (operation 202). At least one of the oligonucleotides may include an abasic site. In some examples, the abasic site is generated by damage to that oligonucleotide. The solution may include a fluorophore coupled to a reactive group. For example, a solution including a suitable solvent (such as water or a buffer) and fluorophore 150 coupled to reactive groups 151 may be flowed over substrate 100 described with reference to FIG. 1A, and oligonucleotide 140 may include abasic site 145 which may be generated by damage to that oligonucleotide.

Method 200 illustrated in FIG. 2 may include reacting the reactive group with the abasic site to couple the fluorophore to the abasic site (operation 204). For example, reactive group 151 may react with aldehyde 143 to couple fluorophore 150 to abasic site 145 in a manner such as described with reference to FIG. 1B. Method 200 illustrated in FIG. 2 may include detecting the abasic site using fluorescence from the fluorophore (operation 206). For example, suitable detection circuitry 160 may detect the fluorescence from fluorophore 150, using which abasic site 145 may be detected. Nonlimiting examples of fluorophore 150 and reactive group 151, and example manners in which nucleotide base(s) adjacent to the abasic site may cause fluorophore 150 to begin to fluoresce, or may enhance fluorescence of fluorophore 150, are described with reference to FIGS. 1A-1B.

Compositions and Methods for Measuring Abasic Sites, Such as Measuring Activity of Glycosylases

It will be appreciated that although examples such as described with reference to FIGS. 1A-1B and 2 may be used to detect unintentionally generated abasic sites on surface-coupled, single-stranded oligonucleotides, the present compositions and methods suitably may be used to detect and, in some examples measure, both intentionally and unintentionally generated abasic sites on any polynucleotide, e.g., polynucleotides that are single-stranded or double-stranded, and that are coupled to a surface (or other element) or are in solution.

Some examples provided herein relate to methods for measuring an amount of abasic sites, such measuring activity of glycosylases. For example, as noted above, glycosylases may be used to intentionally generate abasic sites in polynucleotides, for example to linearize clusters for use in sequencing-by-synthesis. The greater the activity of the glycosylase, the more rapidly the glycosylase generates abasic sites. However, different batches of glycosylase may have different activities than one another, or the activity of a given batch of glycosylase may decrease over time. As such, it may be useful to measure the activity of the glycosylase using a measurement of the amount of abasic sites generated by the glycosylase, e.g., so that the glycosylase may be used for a sufficient amount of time to achieve the desired product, or so that the glycosylase may be discarded if its activity is too low. In some examples, the activity of glycosylases in solution may be measured by coupling fluorophores to abasic sites generated by such glycosylases, and measuring changes as a function of time of the fluorescence from the solution. In some examples, the glycosylase is a monofunctional glycosylase.

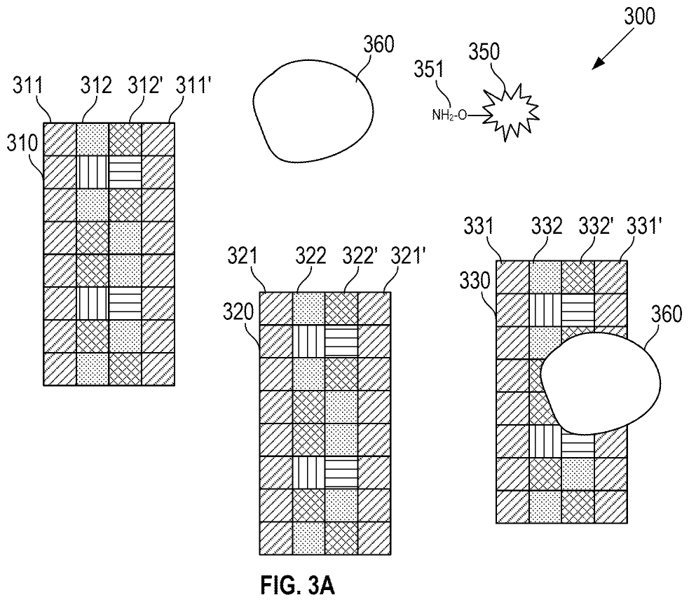

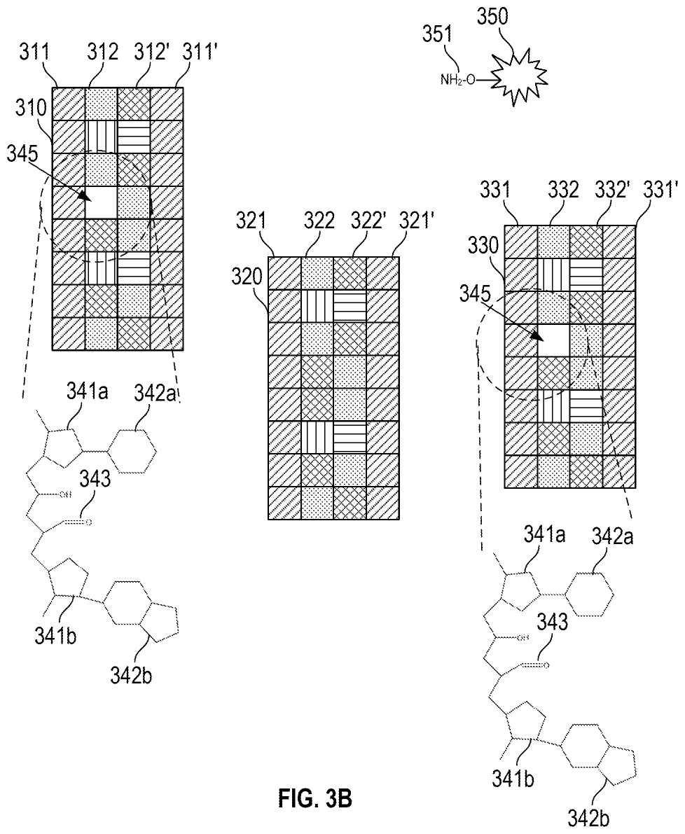

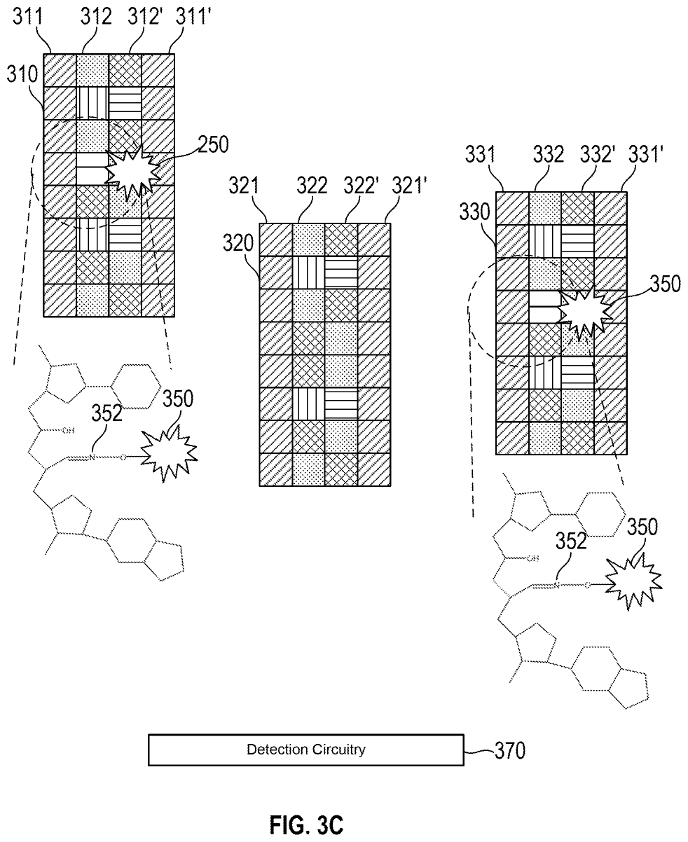

For example, FIGS. 3A-3C schematically an example composition for measuring an amount of abasic sites, such as for measuring glycosylase activity. Composition 300 illustrated in FIG. 3A includes a plurality of oligonucleotides 310, 320, 330 in solution. In the illustrated example, each of oligonucleotides 310, 320, 330 is double-stranded, although it will be appreciated that the oligonucleotides instead may be single-stranded. For example, oligonucleotide 310 includes first sugar-phosphate backbone 311 coupled to first bases 312 and second sugar-phosphate backbone 311′ coupled to second bases 312′ that are hybridized to first bases 312; oligonucleotide 320 includes first sugar-phosphate backbone 321 coupled to first bases 322 and second sugar-phosphate backbone 321′ coupled to second bases 322′ that are hybridized to first bases 322; and oligonucleotide 330 includes first sugar-phosphate backbone 331 coupled to first bases 332 and second sugar-phosphate backbone 331′ coupled to second bases 332′ that are hybridized to first bases 332. In a manner such as suggested by the differently filled boxes in FIG. 3A, the bases 312 of oligonucleotide 310 may have the same sequence as the bases 322 of oligonucleotide 320 and the bases 332 of oligonucleotide 330. However, it will be appreciated that the bases of the oligonucleotides may have any suitable sequence or sequences.

The solution further may include glycosylases 360, fluorophores 350 coupled to reactive groups 351, and a suitable solvent (such as water or a buffer). Oligonucleotides 310, 320, 330 may include abasic sites generated by glycosylases 360 in the solution. The rate at which the glycosylases 360 generate abasic sites depends, in part, on the activity of the glycosylases. For example, at the particular time illustrated in FIG. 3A, a given glycosylase 360 may be acting upon oligonucleotide 330, e.g., using the sequence of that oligonucleotide. At the particular time illustrated in FIG. 3B, the action of glycosylases 360 may have generated abasic sites 345 in oligonucleotides 330 and 310. By a later time (not specifically illustrated), the action of glycosylases 360 upon oligonucleotides may generate further abasic sites 345.

Fluorophores 350 may be coupled to abasic sites 345, and an amount of the abasic sites may be measured using fluorescence from the fluorophores. For example, as illustrated in the inset of FIG. 3B, abasic sites 345 may include aldehydes in a manner such as described with reference to FIG. 1A, and may have a first nucleotide including sugar 341a and (illustratively) pyrimidine base 342a, and a second nucleotide including sugar 341b and (illustratively) purine base 342b, adjacent thereto. As illustrated in FIG. 3C, fluorophores 350 may be coupled to abasic sites 345. For example, fluorophores 350 may be coupled to reactive groups 351 that may react with abasic sites 345 so as to couple fluorophores 350 to abasic site 345 in a manner such as shown in FIG. 3B. Nonlimiting examples of reactive group 351 include hydroxylamine and hydrazine. For example, as shown in the inset of FIG. 3C, hydroxylamines 351 react with aldehydes 343 to form oxime linkages 352 via which fluorophores 350 are coupled to respective abasic sites 345. An amount of the abasic sites 345 may be measured using fluorescence from the fluorophores, e.g., using suitable detection circuitry 370. The activity of the glycosylases 360 may be determined using changes in the intensity of the fluorescence as a function of time. The glycosylases subsequently may be used in another in vitro process, such as an SBS operation (illustratively, but not limited to, linearizing clusters).

In some examples, real-time detection may be achieved if, illustratively, the reaction rate of the fluorophores with abasic sites is faster than the rate at which the glycosylases generate the abasic sites, such that any newly formed abasic sites may couple to respective fluorophores relatively quickly, resulting in turn-on or enhancement of fluorescence. Over time, an increase in the fluorescence may directly correlate with the number of abasic sites that the glycosylases create. The slope of the kinetic curve (fluorescence versus time) may be used to represent the activity of the glycosylase. In other examples, a step-wise detection may be used to compare batch-to-batch activity of the glycosylase. For example, in a first step glycosylases may react with polynucleotides (such as DNA or RNA) to generate abasic sites, followed by a second step in which fluorophores are reacted with the abasic sites to turn-on or enhance fluorescence.

It will be appreciated that fluorophores 350 may include any suitable fluorophores. The nucleotide base(s) adjacent to abasic site 345 may reduce or inhibit nonradiative energy dissipation from fluorophore 350 coupled to that abasic site. For example, fluorophore 350 may include a molecular rotor dye comprising π-conjugated components separated by a rotatable C—C bond. Nucleotide bases 342a, 342b adjacent to the abasic site may restrict rotation of the C—C bond and align the π-conjugated components with one another. Such restriction of rotation, when coupled to abasic site 345, may enhance fluorescence of fluorophore 350 as compared to when in solution, or even may “turn on” fluorescence from fluorophore 350. In some examples, the molecular rotor dye coupled to the reactive group 351 is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange), example structures for which are provided above, in which Z may react with aldehyde 343 so as to form an oxime linkage between fluorophore 350 and abasic site 345.

It will be appreciated that any suitable fluorophore other than a molecular rotor dye suitably may be coupled to a reactive group that may be coupled to an abasic site. Illustratively, the fluorophore coupled to the reactive group 351 may be selected from the group consisting of an Alexa Fluor dye and a 1,8-naphthalene diimide. In one non-limiting example, the Alexa Fluor dye is Alexa Fluor 488, an example structure for which is shown above in which Z may react with aldehyde 343 so as to form an oxime linkage between fluorophore 350 and abasic site 345. In one non-limiting example, the 1,8-naphthalene diimide is 6-dimethylamino)-2-methyl-1H-benzo[de]isoquinoline-1,3(2H)-dione (NP2), an example structure for which is shown above in which Z may react with aldehyde 343 so as to form an oxime linkage between fluorophore 350 and abasic site 345.

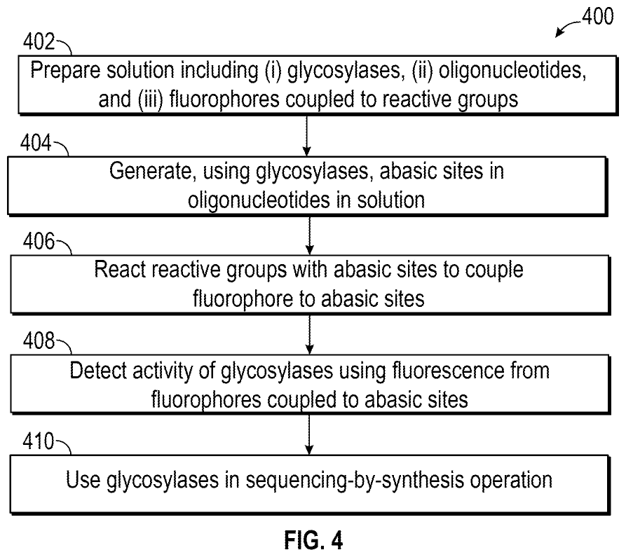

FIG. 4 schematically illustrates operations in an example method for measuring an amount of abasic sites, such as for measuring glycosylase activity. Method 400 illustrated in FIG. 4 may include preparing a solution that includes (i) glycosylases, (ii) oligonucleotides, and (iii) fluorophores coupled to reactive groups (operation 402). For example, a solution may be prepared by mixing together glycosylases 360, oligonucleotides 310, 320, 330, and fluorophores 350 coupled to reactive groups 351, such as described with reference to FIG. 3A, in a suitable solvent, such as water or a buffer.

Method 400 illustrated in FIG. 4 further may include generating, using the glycosylases, abasic sites in the oligonucleotides in the solution (operation 404). For example, in a manner such as described with reference to FIGS. 3A-3B, glycosylases 360 may act upon oligonucleotides 310, 320, 330 and thereby generate abasic sites 345. Method 400 illustrated in FIG. 4 further may include reacting the reactive groups with the abasic sites to couple the fluorophores to the abasic sites (operation 406). For example, in a manner such as described with reference to FIG. 3C, reactive groups 351 may react with abasic sites 345 to couple fluorophores 350 to the abasic sites. Method 400 illustrated in FIG. 4 further may include measuring activity of the glycosylases using fluorescence from the fluorophores coupled to the abasic sites (operation 408). For example, the activity of the glycosylases 360 may be measured using changes in the intensity of the fluorescence as a function of time, e.g., in a manner such as described with reference to FIGS. 3A-3C.

Method 400 illustrated in FIG. 4 further may include using the glycosylases in a sequencing-by-synthesis operation. Illustratively, the glycosylases 360 may be used to linearize amplicons such as may be formed during cluster amplification, e.g., may be used to generate abasic sites at defined locations of the amplicons, following which the backbones of those amplicons may be cut at the abasic sites. It will be appreciated that the glycosylases instead may be used in any other type of operation and are not limited to use in SBS.

### ADDITIONAL EXAMPLES

The following examples are purely illustrative, and not intended to be limiting.

### Example 1. Synthesis of CCVJ1 Hydroxylamine

In one example, the molecular rotor dye CCVJ1 coupled to the reactive group hydroxylamine is synthesized.

Briefly, O-(2-aminoethyl hydroxylamine) protected by tert-butyloxycarbonyl (Boc) is prepared using the following reactions:

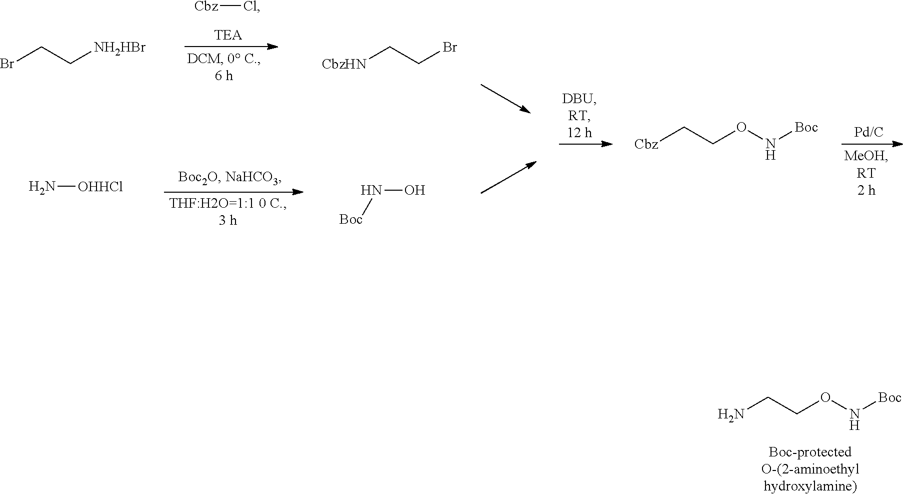

The CCVJ1 core is synthesized via aldol condensation of 2-cyanoacetic acid and 9-formyljulolidine, and then reacted with O-(2-aminoethyl hydroxylamine) deprotected using trifluoroacetic acid (TFA) to obtain CCVJ1 hydroxylamine using the following reactions:

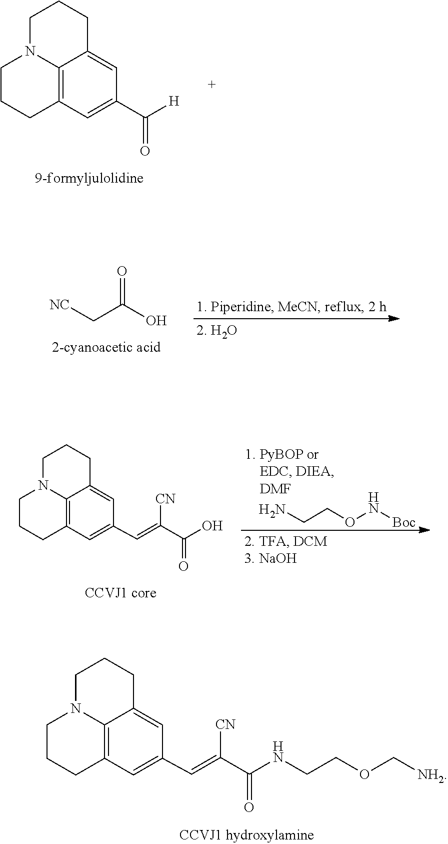 [SMILES: O=Cc1cc2c3c(c1)CCCN3CCC2 ; N#CCC(=O)O ; N#C/C(=C\c1cc2c3c(c1)CCCN3CCC2)C(=O)O ; CC(C)(C)OC(=O)NOCCN ; N#C/C(=C\c1cc2c3c(c1)CCCN3CCC2)C(=O)NCCOCN]

### Example 2. Synthesis of NP2 Hydroxylamine

In another example, the fluorophore NP2 coupled to the reactive hydroxylamine is synthesized.

Briefly, as shown in the reactions below, starting from commercially available 4-bromo-1,8-naphthalic anhydride, the core structure of naphthalene diimide is synthesized via condensation with Boc protected O-(2-aminoethyl)hydroxylamine which is prepared as described in Example 1. Dimethylamine is then installed via nucleophilic aromatic substitution of position 4 bromine. NP2 hydroxylamine is obtained after BoC deprotection using TFA and workup.

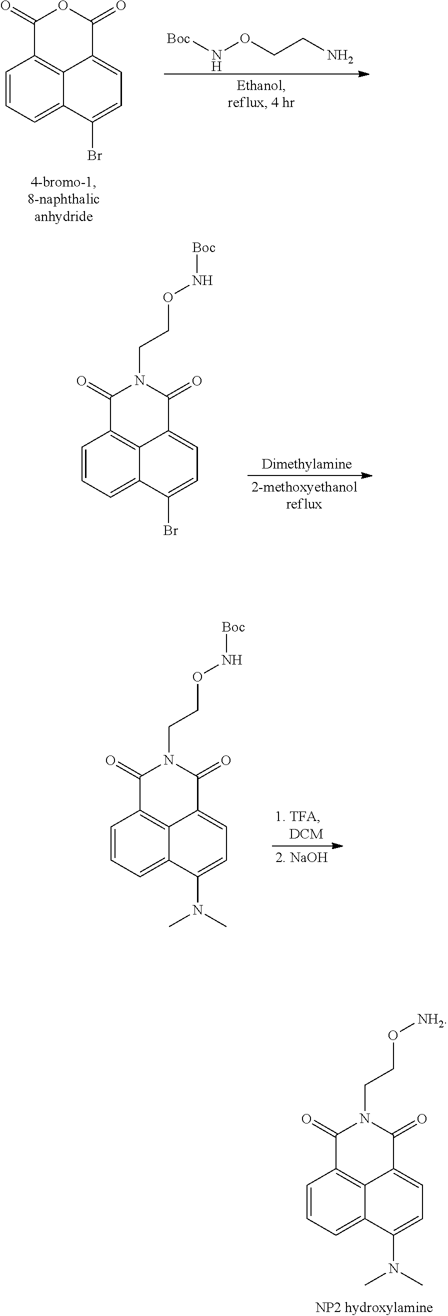 [SMILES: O=C1OC(=O)c2ccc(Br)c3cccc1c23 ; CC(C)(C)OC(=O)NOCCN ; CC(C)(C)OC(=O)NOCCN1C(=O)c2cccc3c(Br)ccc(c23)C1=O ; CN(C)c1ccc2c3c(cccc13)C(=O)N(CCONC(=O)OC(C)(C)C)C2=O ; CN(C)c1ccc2c3c(cccc13)C(=O)N(CCON)C2=O]

### Example 3. Synthesis of DFHBI Hydroxylamine

In another example, the molecular rotor dye DFHBI coupled to reactive group hydroxylamine is synthesized.

Briefly, 4-hydroxy-3,5-difluorobenzaldehyde is condensed with N-acetylglycine in acetic anhydride under reflux. The resulting compound is reacted with deprotected O-(2-aminoethyl) hydroxylamine (see example 1), which converts the oxazole ring into imidazole to obtain DFHBI hydroxylamine as shown in the reaction scheme below:

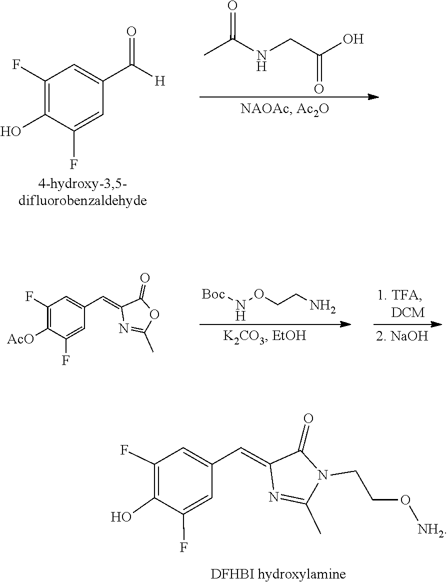

### Example 4. Synthesis of Thiazole Orange Hydroxylamine

In another example, the fluorophore thiazole orange coupled to hydroxylamine reactive group is synthesized.

Briefly, as shown in the reaction scheme below, N substituted quinolone and N substituted benzothiazole compounds are prepared via SN2 reactions with methyl iodide and bromoacetic acid, respectively, and reacted with one another to obtain the thiazole orange core structure which is reacted with Boc (tert-butyloxylcarbonyl) protected O-(2-aminoethyl) hydroxylamine, thendeprotected using TFA (see example 1) to obtain thiazole orange hydroxylamine. In the reaction scheme below, Et3N represents trimethylamine, PyBOP represents benzotriazol-1-yl-oxytripyrrolidinophosphonium hexafluorphosphate (a coupling reagent), EDC represents 1-ethyl-3-(3-dimethylaminopropyl)carbodiimide (an alternative coupling agent), DIEA represents N-N-diisopropylethylamine (a base used in coupling reactions), DMF represents dimethylformamide, and DCM represents dichloromethane.

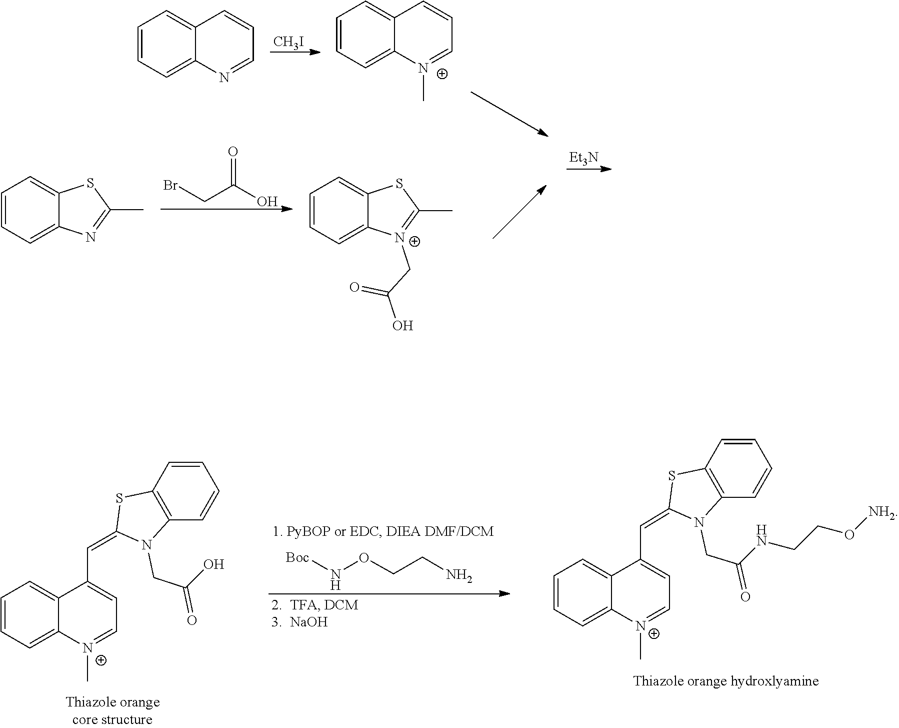

From these examples, it may be understood that different dyes coupled to reactive groups may be synthesized.

### OTHER EXAMPLES

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method for detecting an abasic site, the method comprising:
flowing a solution over a substrate having a plurality of oligonucleotides coupled thereto,
at least one of the oligonucleotides comprising an abasic site,
the solution comprising a fluorophore coupled to a reactive group;
reacting the reactive group with the abasic site to couple the fluorophore to the abasic site; and
detecting the abasic site using fluorescence from the fluorophore.

2. The method of claim 1, wherein the abasic site is generated by damage to the oligonucleotide.

3. The method of claim 1, wherein nucleotide bases adjacent to the abasic site inhibit non-radiative energy dissipation from the respective fluorophore coupled to the abasic site.

4. The method of claim 1, wherein the fluorophore comprises a molecular rotor dye comprising π-conjugated components separated by a rotatable C—C bond.

5. The method of claim 4, wherein nucleotide bases adjacent to the abasic site restrict rotation of the C—C bond and align the π-conjugated components with one another.

6. The method of claim 4, wherein the molecular rotor dye coupled to the reactive group is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange).

7. The method of claim 1, wherein the fluorophore coupled to the reactive group is selected from the group consisting of: 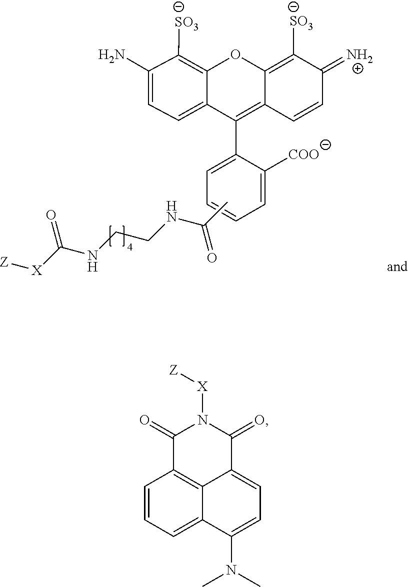
where X is a linker and Z is the reactive group.

8. The method of claim 1, wherein the abasic site comprises an aldehyde.

9. The method of claim 1, wherein the reactive group comprises a hydroxylamine group.

10. The method of claim 1, wherein the reactive group comprises a hydrazine group.

11. The method of claim 1, wherein reacting the reactive group with the abasic site forms an oxime linkage.

12. A composition comprising:
a substrate having a plurality of oligonucleotides coupled thereto,
at least one of the oligonucleotides comprising an abasic site; and
a fluorophore coupled to the abasic site, the abasic site being detectable using fluorescence from the fluorophore.

13. The composition of claim 12, wherein the abasic site is generated by damage to the oligonucleotide.

14. The composition of claim 12, wherein nucleotide bases adjacent to the abasic site inhibit non-radiative energy dissipation from the respective fluorophore coupled to the abasic site.

15. The composition of claim 12, wherein the fluorophore comprises a molecular rotor dye comprising π-conjugated components separated by a rotatable C—C bond.

16. The composition of claim 15, wherein nucleotide bases adjacent to the abasic site restrict rotation of the C—C bond and align the π-conjugated components with one another.

17. The composition of claim 15, wherein the molecular rotor dye coupled to the reactive group is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange).

18. The composition of claim 12, wherein the fluorophore coupled to the reactive group is selected from the group consisting of: 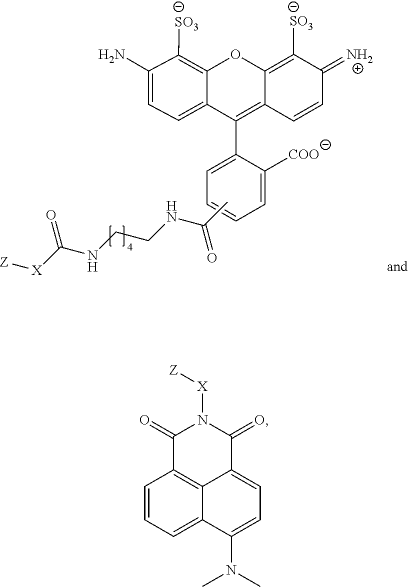
where X is a linker and Z is the reactive group.

19. The composition of claim 13, wherein the abasic site comprises an aldehyde.

20. The composition of claim 13, wherein the reactive group comprises a hydroxylamine group.

21. The composition of claim 13, wherein the reactive group comprises a hydrazine group.

22. The composition of claim 13, wherein reacting the reactive group with the abasic site forms an oxime linkage.

23. A method, comprising:
preparing a solution comprising (i) glycosylases, (ii) oligonucleotides, and (iii) fluorophores coupled to reactive groups;
generating, using the glycosylases, abasic sites in the oligonucleotides in the solution;
reacting the reactive groups with the abasic sites to couple the fluorophores to the abasic sites;
measuring activity of the glycosylases using fluorescence from the fluorophores coupled to the abasic sites; and
using the glycosylases in a sequencing-by-synthesis operation.

24. The method of claim 23, wherein nucleotide bases adjacent to the abasic site inhibit non-radiative energy dissipation from the respective fluorophore coupled to the abasic site.

25. The method of claim 23, wherein the fluorophore comprises a molecular rotor dye comprising π-conjugated components separated by a rotatable C—C bond.

26. The method of claim 25, wherein nucleotide bases adjacent to the abasic site restrict rotation of the C—C bond and align the π-conjugated components with one another.

27. The method of claim 25, wherein the molecular rotor dye coupled to the reactive group is selected from the group consisting of 9-(2-carboxy-2-cyanovinyl)-julolidine (CCVJ1), (Z)-4-(3,5-difluoro-4-hydroxybenzylidene)-1,2-dimethyl-1-H-imidazol-5(4H)-one (DFHBI), and 1-methyl-4-[(3-methyl-2(3H)-benzothiazolylidene)methyl]quidolimium (thiazole orange).

28. The method of claim 23, wherein the fluorophore coupled to the reactive group is selected from the group consisting of: 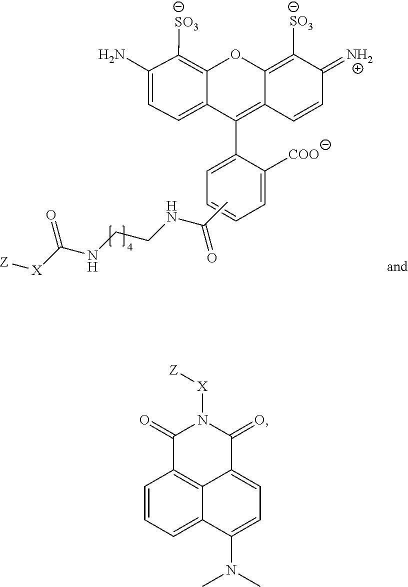
where X is a linker and Z is the reactive group.

29. The method of claim 23, wherein the abasic site comprises an aldehyde.

30. The method of claim 23, wherein the reactive group comprises a hydroxylamine group.

31. The method of claim 23, wherein the reactive group comprises a hydrazine group.

32. The method of claim 23, wherein reacting the reactive group with the abasic site forms an oxime linkage.

33-42. (canceled)


SEQUENCE LISTING (2 sequences)

SEQ ID NO: 1 - DNA, 20 bp, Artificial Sequence
  Description of Artificial Sequence Synthetic primer
aatgatacggcgaccaccga

SEQ ID NO: 2 - DNA, 21 bp, Artificial Sequence
  Description of Artificial Sequence Synthetic primer
caagcagaagacggcatacga